(19) United States
(12) Patent Application Publication
(10) Pub. No.: US 2020/0093397 A1
(43) Pub. Date: Mar. 26, 2020

(54) DETERMINING CATHETER-TIP 3D LOCATION AND ORIENTATION USING FLUOROSCOPY AND IMPEDANCE MEASUREMENTS

(71) Applicant: APN Health, LLC, Pewaukee, WI (US)

(72) Inventors: Jasbir Sra, Pewaukee, WI (US); James Baker, Woodbury, MN (US); Jeffrey Burrell, Coon Rapids, MN (US); Mark Palma, Fitchburg, WI (US); Barry Belanger, Chenequa, WI (US)

(73) Assignee: APN Health, LLC, Pewaukee, WI (US)

(21) Appl. No.: 16/139,966

(22) Filed: Sep. 24, 2018

(51) Int. Cl. A61B 5/06; A61B 6/00; A61B 34/20

(52) U.S. Cl. CPC A61B 5/063; A61B 6/485; A61B 34/20; A61B 5/0452; A61B 6/503; A61B 5/062; A61B 6/487

(57) ABSTRACT

A method for determining the 3D location of a catheter distal end portion in a patient's body, the distal end portion including an electrode, the method comprising: (a) placing first and second body-surface patches on the patient in positions such that body region of interest is therebetween; (b) driving an alternating current between the patches; (c) measuring the voltage at the electrode and substantially contemporaneously capturing a 2D fluoroscopic image of the region of interest; and (d) determining the 3D location of the catheter distal end portion from the image and the measured voltage. A primary application of this method is 3D navigation during cardiac interventional procedures.

### FIELD OF THE INVENTION

The present invention generally relates to medical navigational systems and more particularly to systems for navigation during interventional cardiac and other medical procedures.

### BACKGROUND OF THE INVENTION

Anatomical navigational systems provide the 3D location and orientation of a navigational catheter within a cardiac chamber of interest and, in some instances, can also be used to construct 3D maps of the cardiac chamber. Most of these systems are, however, quite expensive to both acquire and operate, and consume substantial clinician and technician resources for setup and operation. Some of these systems require specifically-designed catheters, such as catheters with built-in sensors, which are in themselves expensive.

For example, there are several USFDA-cleared 3D cardiac mapping systems currently in use. Among these are Biosense Webster's CARTO® system and St. Jude Medical's (now owned by Abbott Laboratories) EnSite™ NavX™ system. These systems utilize expensive hardware and software platforms and require expensive and proprietary catheters with built-in sensors or custom patch sets. Furthermore, due to their complexity, their operation typically requires highly-trained application specialists. Therefore, such systems, while effective, are available only in a limited number of medical and research facilities for use during interventional procedures.

Biplane fluoroscopy provides another method for improved cardiac visualization, but it is also relatively expensive, increases radiation exposure to the patient, and is also not commonly available in electrophysiology (EP) labs. Due to these several limitations, important cardiac interventional procedures such as cardiac ablation are not readily available to many patients who suffer from cardiac arrhythmia.

Conventional fluoroscopy systems, on the other hand, are available in essentially all cardiac interventional labs for imaging and real-time navigation of electrophysiology (EP) catheters and other instruments and for the placement of leads and stents during interventional procedures. Other than the initial acquisition cost, such systems require little ongoing operational cost. Further, conventional fluoroscopic systems are able to visualize any type of catheter. However, these systems alone do not provide the 3D visualization that is essential for mapping and ablation of cardiac arrhythmia. In a typical fluoroscopic image taken during a procedure, it is only possible to view catheter location along the x-y plane; the z-axis (depth) is not discernible. Thus, there is no depth perception in the 3D space where the cardiac structures are being mapped.

Recently, APN Health®, LLC has developed its Navik 3D® system, the basics of which are disclosed in U.S. Pat. No. 9,986,931 (Sra et al.) titled “Automatically Determining 3D Catheter Location and Orientation Using 2D Fluoroscopy Only,” and the entire document is included herein by reference.

The Navik 3D® system uses real-time two-dimensional (2D) fluoroscopic images from single-plane fluoroscopy systems and body-surface electrocardiogram (ECG) and intracardiac electrogram (EGM) signals from patient recording and monitoring systems to create and display 3D maps of the cardiac chamber of interest. This process does not require special catheters or dedicated technicians, and is appropriately operated using fluoroscopy at accepted standards of care. The Navik 3D® system may be used as an additional resource to existing EP lab equipment such as conventional fluoroscopy and patient recording and monitoring systems. The live images and signals from each of these systems remain available for the operator throughout Navik 3D® use and do not experience interference from the operation of the Navik 3D® system.

The foundational ideas behind the Navik 3D® system disclosed in the above-mentioned Sra et al. patent are (1) the recognition that the 2D projection of a single-plane fluoroscopic image contains information about the position of the object in 3D and (2) the application of “pixel-level geometric calculations” to achieve the accuracy required given the constraints of image resolution within single-plane fluoroscopic images. Extracting z-axis (the third or depth dimension in an x,y,z coordinate system) information from fluoroscopic images involves the application of X-ray conic projection and physics principles using software algorithms to generate the 3D location of the catheter from these 2D images. The 3D position of a catheter tip is determined based on the detected (magnified) size of the catheter tip in the fluoroscopic image, the known distance from the X-ray source to the fluoroscopy detector, and the known width of the catheter tip determined from an initialization process.

Pixel-level geometric calculations as defined in Sra et al. refer to calculations which preserve the original pixel-intensity values and permit statistical calculations to be performed on the pixel intensity values. Meaningful statistical analysis can be performed on such data since the pixel intensities are not transformed by filters. (The application of filters to image data changes pixel-intensity values in the filtered images and therefore causes some loss of information from the image data.) The result of using the unfiltered data and statistical analysis is that useful sub-pixel accuracy can be achieved. In fact, the data from many conventional fluoroscopes are close enough to “raw data” such that the “one over the square root of n” improvements in accuracy do occur (n being the number of statistically-combined profiles). Consequently, the Navik 3D® system based on the disclosure in the Sra et al. patent has matched or bettered the accuracy of other much more costly systems.

In certain applications, however, it is sometimes desirable to limit the X-ray exposure of a patient below levels which may be necessary with the Navik 3D® system or to “see” a catheter in positions in which it may be difficult to extract the third dimension effectively with Navik 3D®. It may also be helpful to map or track a catheter at speeds faster than those achievable with Navik 3D®.

Systems which utilize measurements of electrical impedance between catheter electrodes and body-surface patches to determine a 3D relative position estimate such position by examining the changes in impedance across multiple axes. This is generally achieved using multiple body-surface patches placed across the patient to enable impedance readings across multiple axes to achieve an estimate of a 3D spatial coordinate set.

Magnetic tracking is another technique which is used to navigate catheters in a patient's body. Systems using this technique require placement of electrical coils under the patient and special catheters in which coils are embedded. Magnetic fields produced by the electrical coils under the patient are measured by sensor coils in the catheter. Not only are the specialized catheters expensive, but other challenges are found in such systems, such as (a) tracking can be susceptible to metallic changes near the patient, including movement of the C-arm of a fluoroscope and (b) calibration of the system to accommodate movement of the C-arm is often complicated.

All major cardiac mapping systems use some sort of a hybrid approach to provide catheter localization. Biosense Webster's CARTO® system utilizes a magnetic system as its primary modality and augments the magnetic system with a impedance measurement subsystem. The localization methods for both St. Jude's Ensite™ NavX™ system and Boston Scientific's Rhythmia HDx™ system are impedance measurements augmented by a magnetic subsystem. In each of these products, the impedance subsystems are three-dimensional systems using impedance measurements for determining location in all three dimensions.

As mentioned above, such systems are both complex and costly and as such, there is a need for a much more cost-effective cardiac navigational system, in particular one that can be adopted by a much larger number of hospitals around the world. In addition, a patient undergoing a procedure with one of these systems typically receives some level of X-ray exposure since these systems often use fluoroscopy for confirmation of catheter-tip location.

Conventional fluoroscopic systems have an important technical advantage in that measurement accuracy within a single-frame fluoroscopic image is very high in the plane (herein sometimes referred to as the x,y plane) of the fluoroscopic detector. For a typical detector with resolution of 1000×1000 pixels and an area of 20×20 cm, the pixels are spaced 0.2 mm apart, and although there are sources of noise such as X-ray quantum statistical noise, such a geometric arrangement provides high accuracy in the detector plane. The Navik 3D® system discussed above requires multiple fluoroscopic images to determine the third dimension (herein referred to as the z-coordinate, z-dimension, depth or depth dimension), and such multiple fluoroscopic images are the cause of X-ray exposures being high in certain applications of the Navik 3D® system.

Thus, there is a need for a cardiac navigational system which exploits the high geometric accuracy of fluoroscopic images in the two dimensions of the X-ray detector plane while capturing the third spatial dimension in a fashion which is both rapid and limits the X-ray exposure of a patient. The invention disclosed herein is a hybrid system which combines 2D fluoroscopy to capture two spatial dimensions and measurement of the electrical impedance within a cardiac chamber of patient's torso to capture the third spatial dimension.

This and other objects of the invention will be apparent from the following descriptions and from the drawings.

It should be appreciated that although applicable to other regions of a body, the present invention is described with particular reference to 3D navigation during a cardiac interventional procedure.

### SUMMARY OF THE INVENTION

The invention disclosed herein is a method for determining the 3D location and orientation of a catheter tip in a patient's cardiac chamber. The catheter has a distal end portion (sometimes herein referred to as a catheter tip) and two or more electrodes adjacent to the distal end. The method includes the steps of: (a) placing first and second body-surface patches on the patient in locations such that the cardiac chamber is between the first and second body-surface patches, the first and second body-surface electrodes defining a depth dimension; (b) driving an alternating current between the patches; (c) measuring the voltage at the electrodes and substantially contemporaneously capturing a 2D fluoroscopic image of the cardiac chamber; and (d) determining the 3D location and orientation of the catheter distal end portion from the image and the measured voltages.

Some preferred embodiments of the method include placing a body-surface reference patch on the patient, the voltages being measured with respect to the reference patch.

Some preferred embodiments have one or more of the following features: the alternating current has a constant peak-to-peak amplitude; the first body-surface patch is positioned on the patient's chest, and the second body-surface patch is positioned on the patient's back; and the step of measuring voltage includes using synchronous detection. In some of these embodiments, the step of measuring voltage includes applying a Goertzel filter to the voltage. Further, in some embodiments, the output of the Goertzel filter is a complex number having real and imaginary parts, and the output is transformed into a real number by computing the square root of the sum of the squares of the real and imaginary parts, and in some of these embodiments, a window function is applied to the voltage prior to applying the Goertzel filter. In some embodiment, the window function is a Blackman window.

Some preferred embodiments of the inventive method include correcting for changes in fluoroscopic table position and orientation and C-arm angle.

Some highly-preferred embodiments include the calibration steps of (i) locating one electrode of the catheter distal end portion at two or more calibration locations within the cardiac chamber, some of the calibration locations being separated from the other calibration locations along the depth dimension; (ii) determining spatial coordinates of the one electrode in each calibration location using only fluoroscopy; (iii) measuring the voltages at the one electrode at each calibration location; and (iv) computing a depth-versus-voltage relationship therefrom. In some of these embodiments, determining the spatial coordinates of the one electrode includes capturing two 2D fluoroscopic images of the cardiac chamber from different angles and applying back-projection calculations thereto. In some of these embodiments, determining the spatial coordinates of the one electrode includes the steps of: (1) capturing a stream of digitized 2D images of the cardiac chamber from a single angle; (2) detecting an image of the one electrode in a subset of the digital 2D images; (3) applying to the digital 2D images calculations which preserve original pixel intensity values and permit statistical calculations thereon, using a plurality of unfiltered raw-data cross-sectional intensity profiles and statistically combining the profiles to estimate image dimensions, thereby to measure the electrode image; (4) applying conical projection and radial elongation corrections to the image measurements; and (5) calculating the spatial coordinates of the electrode from the corrected 2D image measurements.

In some highly-preferred embodiments, computing the depth-versus-voltage relationship includes determining a linear regression relationship between the voltages and the corresponding depths of the calibration locations.

Some highly-preferred embodiments include placing a body-surface impedance-monitoring patch on the patient, measuring the voltage thereon, and monitoring bulk impedance of the patient. Some of these embodiments include the step of recalibration when a change in the bulk impedance exceeds a threshold.

In some preferred embodiments of the inventive method, measuring the voltages and capturing the 2D fluoroscopic images are gated by respiratory phase, and in some embodiments, measuring the voltages and capturing the 2D fluoroscopic images are gated by cardiac phase.

In some preferred embodiments, one of the two or more electrodes is an ablation electrode, and the ablation electrode is electrically-isolated from voltage measurement circuitry during ablation.

Some highly-preferred embodiments of the inventive method include capturing ECG/EGM signals from the patient and time-marking the measured voltages, the captured 2D fluoroscopic image, and the ECG/EGM signals with a common timing signal. Some of these embodiments also include time-marking a respiration signal with the common timing signal.

In another aspect of the inventive method for determining the 3D location of a catheter distal end portion in a patient's body, the distal end portion including an electrode, the method comprises: (a) placing first and second body-surface patches on the patient in positions such that a body-region of interest is therebetween; (b) driving an alternating current between the patches; (c) measuring the voltage at the electrode and substantially contemporaneously capturing a 2D fluoroscopic image of the region of interest; and (d) determining the 3D location of the catheter distal end portion from the image and the measured voltage.

### BRIEF DESCRIPTION OF THE DRAWINGS

FIG. 1 is a block diagram schematic of an embodiment for performing the steps of the inventive method for determining the 3D location and orientation of a catheter tip in a cardiac chamber of a patient using both fluoroscopic image data and single-axis electrical impedance data.

FIG. 2 is a schematic representation of the geometry of a fluoroscopic system.

FIG. 3 is a schematic representation of the geometry of a fluoroscopic system as configured for the determination of the 3D coordinates of an object using back-projection.

FIG. 4 is a schematic representation of an embodiment of the single-axis impedance system for determining the depth coordinate of a catheter tip in a cardiac chamber of a patient. FIG. 4 is also used to describe one embodiment of a calibration method for such system.

FIG. 5 is a drawing of a catheter tip as represented in FIG. 4.

FIG. 6A is a simplified electrical circuit model describing the operation of the single-axis impedance system embodiment of FIG. 4.

FIG. 6B is a table illustrating exemplary values within the electrical circuit model of FIG. 6A.

FIG. 7A is a schematic representation of the single-axis impedance system embodiment of FIG. 4 illustrating an embodiment of an alternative calibration method.

FIG. 7B is a schematic illustration of an enlarged portion of the single-axis impedance system embodiment of FIG. 4, illustrating an embodiment of a variant of the alternative calibration method for FIG. 7A.

FIG. 8 is a plot illustrating the alternative calibration methods of FIGS. 7A and 7B.

FIG. 9A is a functional block diagram of an embodiment of the single-axis impedance system for determination of the depth coordinate of a catheter tip in a cardiac chamber of a patient.

FIG. 9B describes an embodiment of a Goertzel filter for which the input voltage has been windowed using a Blackman window.

FIG. 10 is a block diagram schematic illustrating an embodiment of a method for substantially contemporaneously measuring voltages and capturing images, and in this embodiment, gating these steps by both cardiac and respiratory phase to reduce the motion within the fluoroscopic images.

FIGS. 11A-11D are illustrations of exemplary cardiac and respiratory signals being combined to generate a gating signal for the embodiment of FIG. 10. FIG. 11A illustrates an exemplary cardiac signal showing two local activations (R-waves).

FIG. 11B illustrates the exemplary cardiac signal of FIG. 11A but with twelve local activations (R-waves) occurring rapidly such as when a patient is experiencing atrial fibrillation.

FIG. 11C illustrates an idealized exemplary respiration signal from a sensor for measuring respiration phase; FIG. 11C shows one breathing cycle.

FIG. 11D is a schematic representation of a gating signal generated by combining the cardiac and respiration signals.

FIG. 12 is an idealized representation of the variation of bulk impedance across a portion of the chest of a patient.

### DETAILED DESCRIPTION OF PREFERRED EMBODIMENTS

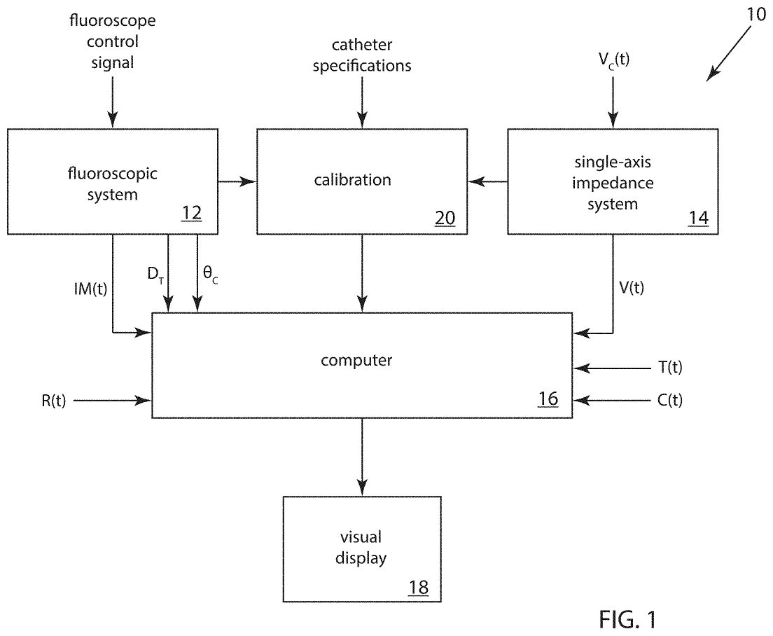

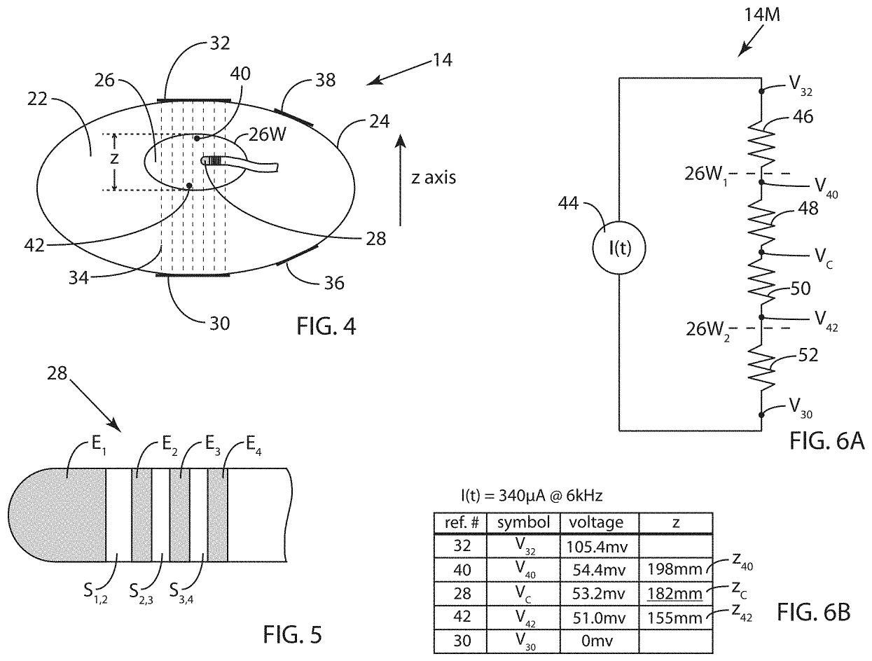

FIG. 1 is a block diagram schematic of an embodiment 10 for performing the steps of the inventive method for determining the 3D location and orientation of a catheter tip 28 (see FIG. 4) in a patient's cardiac chamber 26 using both fluoroscopic image data and single-axis electrical impedance data. (Both the system structure and the method steps are herein referred to as embodiment 10.) Embodiment 10 involves flows of various forms of data and signals including single-plane fluoroscopic images IM(t) of cardiac chamber 26 from a fluoroscopic system 12, voltage V(t) processed by and output from a single-axis electrical impedance system 14, body-surface electrocardiogram (ECG) and intracardiac electrogram (EGM) signals C(t) from patient cardiac recording and monitoring systems (not shown), a respiration signal R(t) indicating respiration phase from a respiration measurement system (not shown), and a timing signal T(t) providing reference timing by which the signals within embodiment 10 are synchronized.

A programmable computer 16 configured and programmed to carry out the steps of embodiment 10 receives the aforementioned data and signals and provides numerical and graphical information to at least a visual display 18 which presents to the electrocardiologist the 3D and other pertinent information by which to carry out a cardiac interventional procedure such as cardiac ablation.

Other data is available to computer 16 such as a C-arm angle θ_{C} and fluoroscopic table position and/or orientation D_{T} from fluoroscopic system 12 indicating the position/orientation of the X-ray beam relative to a patient, catheter specifications such as catheter type/model and geometric data describing catheter tip 28, and calibration data from a calibration process 20. Fluoroscopic system 12 may also provide signals containing table data D_{T} which provides information on the position and orientation of the fluoroscope table (not shown). Calibration process 20 is indicated as a separate block in FIG. 1; although its method steps are carried out within computer 16, calibration process 20 operates only periodically and is thus shown separately from computer 16 in FIG. 1.

Note that in embodiment 10, many of the signals indicated may be digitized signals. Herein, many analog and digital signals are indicated for simplicity as functions of time t (e.g., f(t)) rather than using a time index for streams of digital signals. Digital signals will be explicitly indicated as such in their descriptions. For example, as will be described later, catheter electrode signal V_{C}(t) is an analog signal captured by an electrode while V(t) is a digital stream of values output from single-axis impedance system 14. Fluoroscopic image stream IM(t) is a stream of two-dimensional arrays of digital image-intensity values captured by an X-ray detector D within fluoroscopic system 12.

As described above, the inventive hybrid fluoroscopic/impedance navigational method exploits the high geometric accuracy of fluoroscopic images in the two dimensions of the plane of X-ray detector D while rapidly capturing the third spatial dimension (depth) in a fashion which limits the X-ray exposure of a patient, combining 2D fluoroscopy to capture two spatial dimensions and measurement of the electrical impedance of and within a patient's torso 22 (see FIG. 4) to capture the third spatial dimension (depth). Other significant advantages of the present inventive method are discussed later in this document.

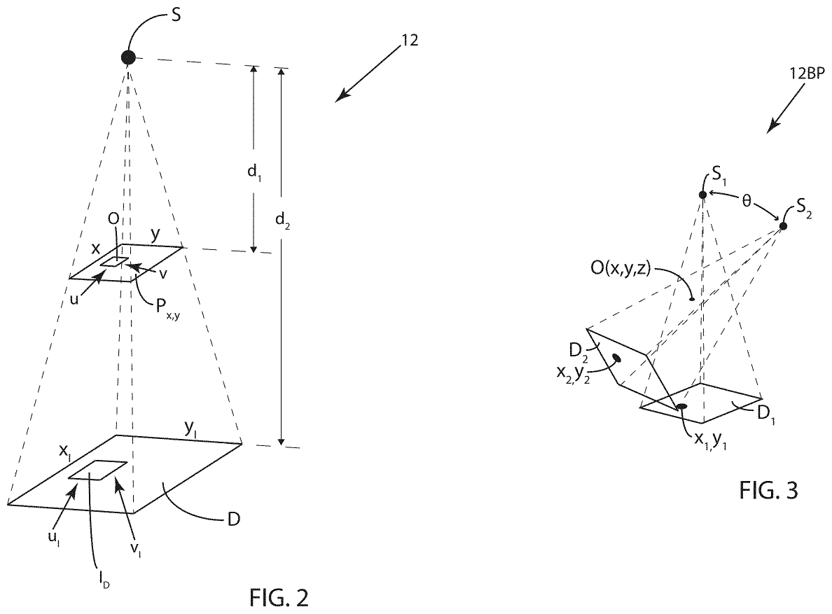

FIG. 2 is a schematic representation of the geometry of fluoroscopic system 12. As is well-known, an X-ray source S emits X-ray radiation in the form of a cone onto an X-ray detector D at a source-to-detector distance of d₂. The X-ray beam passes through the patient, being absorbed by various amounts in the patient's body tissue and X-ray opaque objects such as catheter tip 28. Such an object O is illustrated in FIG. 2 as being in a plane P_{x,y} at a source-to-object distance d₁, and it is this distance d₁ (depth) which is determined by single-axis impedance system 14.

In FIG. 2, object O has x,y dimensions u,v, respectively, while due to the geometry of fluoroscopic system 12, an image I_{D} of object O in the plane of detector D has x_{I},y_{I} dimensions of u_{I},v_{I}, respectively. From simple trigonometric considerations, dimension v is simply equal to v_{I}·d₁/d₂, and dimension u is simply equal to u_{I}·d₁/d₂. If a value for depth dimension d₁ of object O is known from another measurement, in this case from single-axis impedance system 14, then the x,y dimensions of object O can be determined with considerable accuracy from a single image I_{D}. The accuracy with which the dimensions (and coordinates) in plane P_{x,y} can be determined depends on the structure of detector D and other factors such as quantum noise and also on the accuracy with which d₁ is determined. As described above, fluoroscopic system 12 may have a typical pixel-to-pixel distance of 0.2 mm in the plane of detector D. Thus, even with only modest accuracy in the determination of distance d₁, fluoroscopic system 12 has more than ample accuracy in each X-ray image for determination of two of the three dimensions being determined.

By comparison, the aforementioned system for determining 3D catheter location and orientation using only 2D fluoroscopy disclosed in the Sra et al. determines d₁ from writing the above relationship as d₁=d₂·v/v_{I}. The calculation of depth d₁ of catheter tip 28 from its width v_{I} in image I_{D} is very sensitive to the determination of width v_{I}. For a 7 French catheter (2.33 mm diameter) and typical imaging geometry for fluoroscopic system 12, achieving a depth accuracy of approximately +4 mm requires measurement accuracy of width v_{I} of approximately 0.02 mm. Such measurement accuracy is subpixel, and in order to achieve such subpixel accuracy using width measurement from a pair of edge points in image I_{D}, the error required for each edge point is 0.02 mm/2=0.01 mm. The fraction of a pixel corresponding to a precision of 0.01 mm is 0.01/0.2=0.05 pixels or about 1/20th of a pixel. Therefore, in order to achieve this accuracy of depth d₁, the Sra et al. approach incorporates statistical calculations of many width measurements and the use of multiple images.

FIG. 4 is a schematic representation of an embodiment of single-axis impedance system 14 for determining the depth coordinate of catheter tip 28 in cardiac chamber 26. A torso 22 of a patient is shown having a body surface 24. Cardiac chamber 26 having a chamber wall 26W is within torso 22, and catheter tip 28 within cardiac chamber 26. FIG. 5 is a magnified representation of catheter tip 28. Catheter tip 28 has a distal end electrode E₁ (an electrode which may be used for both voltage measurement and tissue ablation) and three electrodes E₂, E₃, and E₄ adjacent to tip E₁. Electrodes E₂, E₃, and E₄ are spaced apart by interelectrode spaces S_{1,2}, S_{2,3}, and S_{3,4}. The dimensions and spacings of these electrodes are at least a portion of the catheter specifications which constitute known data provided to embodiment 10 as indicated in FIG. 1.

Referring again to FIG. 4, a first body-surface patch 30 is shown placed on the back of body surface 24 of torso 22, and a second body-surface patch 32 is shown placed on the chest of body surface 24 of torso 22 such that cardiac chamber 26 is between first 30 and second 32 body-surface patches. Body-surface patches 30 and 32 span across a region which defines a single dimension herein called depth, the depth dimension, the z-dimension, or the third spatial coordinate. An alternating current is driven across the gap between body-surface patches 30 and 32, resulting in an alternating electric field 34 represented by seven dotted lines between body-surface patches 30 and 32. In other words, the depth dimension z is a measurement of the position along the axis defined by body-surface patches 30 and 32 and parallel to alternating electric field 34. Embodiment 10 also includes a body-surface reference patch 36 which provides the reference electrode relative to which all of the voltages in embodiment 10 are measured. Also, embodiment 10 includes a body-surface impedance patch 38, the function of which will be discussed later in this document. Body-surface patches 30, 32, 36 and 38 may be similar to those used for transcutaneous electrical nerve stimulation (TENS), typically consisting of a foam substrate, conductive layer and hydrogel. The conductive layer includes a conductive carbon-film connected to an lead wire. Such specific body-surface patches are not intended to be limiting; any suitable patch may be employed.

FIG. 6A is a simplified electrical circuit model 14M describing the function of impedance system embodiment 14 of FIG. 4. An alternating current source 44 provides an alternating current I(t) through torso 22, including cardiac chamber 26, causing alternating electric field 34 in the region in which voltage measurements are made. The model of FIG. 6A is a simplification since electric field 34 is not quite as simple as illustrated therein due to electrical behavior of the various types of tissue encountered by electric field 34 since the current which flows through the various types of tissue differs. However, since cardiac chamber 26 contains blood, within that small region, it can be assumed that within a plane perpendicular to electric field 34, the impedance remains constant and thus the simplified model sufficiently describes the electrical behavior of electric field 34.

FIG. 6B is a table illustrating exemplary values within electrical circuit model 14M of FIG. 6A and will be used below to illustrate the function of single-axis impedance system 14.

It should be noted that although single-axis impedance system 14 is indeed an electrical impedance-based system, all of the measurements being made are of voltages and the values of the various impedances involved need not be determined. (In the model of FIG. 6A, values of impedances 46, 48, 50 and 52, illustrated in the simplified model as resistors, are not shown although for the example of table of FIG. 6B, resistors 46 and 52 are assumed to have resistance values of 150 ohms, and the sum of resistors 48 and 50 is a resistance of 10 ohms.)

It should also be noted that an alternating current is employed to minimize the nonlinear effects of the interface between electrodes and the conductive fluids in a human body. Voltage measurements are peak-value measurements.

Electrical behavior of the simplified circuit model of FIG. 6A is well-known by those skilled in electrical engineering and need not be described in more detail. However, the result of such a circuit configuration is that if the voltages V₄₀ and V₄₂ and the values of the depth (along the z-dimension as indicated in FIG. 6A) are known at points 40 and 42, then voltage V_{C} in cardiac chamber 26 at, for example, electrode E₂ of catheter 28, determines the depth coordinate of electrode E₂.

Referring to both FIGS. 6A and 6B, as electrode E₂ is moved in depth dimension between known points 40 and 42, the value of the depth z_{C} of electrode E₂ varies linearly with measured voltage V_{C}. The relationship is as follows:

z_{C}=[(z₄₀−z₄₂)/(V₄₀−V₄₂)]·(V_{C}−V₄₂)+z₄₂.

Rewriting this depth-versus-voltage relationship results in a relationship: z_{C}=A·V_{C}+z₄₂ where A is a scalar-valued scale factor in units of mm/mv (millimeters/millivolt). Note that with constant peak-to-peak current I(t), impedance is proportional to voltage so that scale factor A can also be determined in units of millimeters/ohm (mm/Ω).

In the description above, the z-coordinates of points 40 and 42 have been assumed to be known in the calculations of scale factor A and depth z_{C}. These values are known as a result of a calibration method in which the z-coordinates of an electrode (e.g., electrode E₂) are determined by locating electrode E₂ at two or more calibration locations within cardiac chamber 26 at which these calibration locations are separated from the other calibration locations along z-dimension. (Such use of electrode E₂ for this and in later descriptions is exemplary and is not intended to be limiting; any electrode may be used.) Then fluoroscopic system 12 is used to determine the spatial coordinates of electrode E₂ in each calibration location while substantially contemporaneously capturing voltages at electrode E₂. This information is then used to compute a depth-versus-voltage relationship as described above.

Three approaches to calibration are disclosed in this document. The first of these has already been described above with respect to FIG. 6A. In such a calibration procedure, points 40 and 42 (electrode positions) are located as well as possible at points near the top (anterior) and bottom (posterior) of cardiac chamber 26, respectively, and the 3D positions of electrode E₂ are determined using fluoroscopic systems 12 (see below). Scale factor A is then determined using the relationship for A presented above.

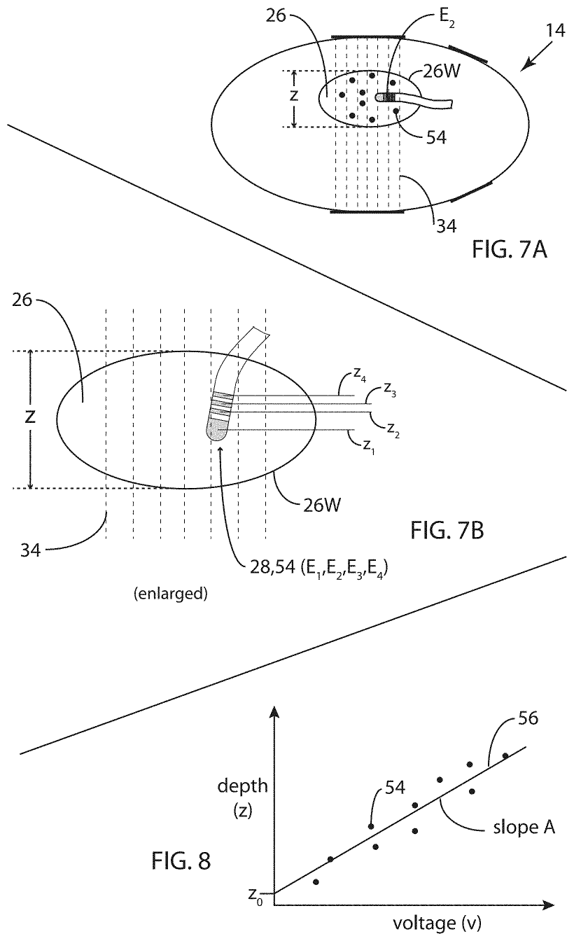

A second approach to calibration is illustrated in FIGS. 7 and 8. FIG. 7A is a schematic representation of impedance system embodiment 14 illustrating an embodiment of an alternative calibration method. As shown in FIG. 7A, electrode E₂ is located at a number of points 54 within cardiac chamber 26 such that a variety of z-coordinate values are represented in the group of points 54. As before, fluoroscopic system 12 is used to determine the 3D location of electrode E₂, and in particular, the z-coordinate of each location 54. These measurements are illustrated in the exemplary plot of FIG. 8 in which the group of points 54 are plotted as z-coordinate versus voltage, and straight line 56 is computed by linear regression on point 54 in order to determine the depth-versus-voltage relationship z=A·v+z₀ represented by line 56.

During calibration, determination of the 3D location of an electrode using only fluoroscopy may be done in at least two ways. A first method includes determining the spatial coordinates (x,y,z) of electrode E₂ at two locations in cardiac chamber 26 by capturing for each of the two points two 2D fluoroscopic images of cardiac chamber 26 (and electrode E₂) from different angles and applying back-projection calculations thereto. The details of back-projection calculations are well-known to those skilled in the area of mathematics and will not be described here. Nevertheless, by way of illustration, FIG. 3 schematically illustrates the geometry of fluoroscopic system 12BP (fluoroscopic system 12 used in back-projection mode) with the angle difference θ between the two C-arm positions such that 2D measurements x₁,y₁ in detector plane D₁ and x₂,y₂ in detector plane D₂ are sufficient to mathematically resolve the 3D location of electrode E₂ in the two locations in cardiac chamber 26.

Referring again to FIG. 4 as well as FIG. 3, a fluoroscopic image is captured with electrode E₂ at point 40 and the C-arm of fluoroscopic system 12 positioned such that X-ray source S is represented by source S₁ and detector D is represented by detector D₁. Then a fluoroscopic image is captured with electrode E₂ at point 40 and the C-arm of fluoroscopic system 12 positioned such that X-ray source S is represented by source S₂ and detector D is represented by detector D₂.

Following this capture of two fluoroscopic images of electrode E₂ from different angles, electrode E₂ is moved to point 42 and two fluoroscopic images of electrode E₂ at point 42 are captured from different angles, this time first with fluoroscopic system 12 configured at source S₂ and detector D₂ and then at source S₁ and detector D₁. Now, with x,y-coordinate pairs x₁,y₁ and x₂,y₂ measured for each of points 40 and 42, there is sufficient data to determine the 3D coordinates of both points 40 and 42 using back-projection calculations.

A voltage measurement is taken substantially contemporaneously with the capture of each of the images such that voltage measurements are known as best as possible at the times of image capture. Also, gating with cardiac phase and/or with respiratory phase may be employed so that not only blurring within the fluoroscopic images is minimized but so that, as best as possible, the 3D coordinates of each point 40 (and 42) when taken at different times, are the same from different C-arm angles.

An alternative method for determining the 3D location of an electrode during calibration is described in detail in the aforementioned Sra et al. reference. This alternative method includes the steps of: (a) capturing a stream of digitized 2D images of cardiac chamber 26 from a single C-arm angle θ_{C}; (b) detecting an image of electrode E₂ in a subset of the digital 2D images; (c) applying to the digital 2D images calculations which preserve original pixel intensity values and permit statistical calculations thereon, using a plurality of unfiltered raw-data cross-sectional intensity profiles and statistically combining the profiles to estimate image dimensions, thereby to measure the image of electrode E₂; (d) applying conical projection and radial elongation corrections to the image measurements; and (e) calculating the spatial coordinates of the electrode from the corrected 2D image measurements. As stated above, the use of electrode E₂ is exemplary in this description and not intended to be limiting. Also note that initialization of the method described in the Sra et al. reference requires a back-projection process prior to the above operations.

In this alternative method, the C-arm angle θ_{C} of fluoroscopic system 12 remains unchanged during calibration, and the 3D location of electrode E₂ is determined at two or more positions within cardiac chamber 26. Calibration may be carried out as illustrated in FIG. 4 using two locations of electrode E₂ or may be carried out at several more locations as illustrated in FIG. 7A (ten locations shown including that on catheter tip 28). At each such point, the third dimension (the depth dimension) is found from the method steps outlined above and employed in the computation of a depth-versus-voltage relationship as described above.

Again as above, a voltage measurement is taken substantially contemporaneously with the capture of each of the images such that voltage measurements are known as best as possible at the times of image capture, and gating with cardiac phase and/or with respiratory phase may be employed.

FIG. 7B is a schematic illustration of a portion of single-axis impedance system 14 as embodied in FIG. 4, illustrating an embodiment of a variant of the alternative calibration method for FIG. 7A. FIG. 7B is an enlargement of such portion, showing cardiac chamber 26 and chamber wall 26W, alternating electric field 34, and catheter tip 28 having four electrodes E₁, E₂, E₃, and E₄ as illustrated in FIG. 5. In this variant of the alternative method described using FIG. 7A, catheter tip 28 is aligned as well as possible with electric field 34, and the four electrodes E₁, E₂, E₃, and E₄ are four points 54 as in FIGS. 7A and 8. In this way, a single fluoroscopic measurement cycle (e.g., by back-projection cycle or by that of the Sra et al. reference) is used to determined the corresponding depths z₁, z₂, z₃, and z₄ as illustrated in FIG. 7B. Scale factor A is then found using the available points 54 from this calibration method as illustrated in FIG. 8. Additionally, this variant embodiment of the alternative calibration method can be applied to more than one fluoroscopic measurement cycle such that, for example, if the catheter being used has four electrodes as illustrated in FIGS. 5 and 7B, then for each such measurement cycle, four calibration points are generated, and in three such cycles, twelve calibration points are generated.

During normal operation of method embodiment 10, in order to determine the orientation of catheter tip 28 as well as its location, voltage measurements are made at more than one electrode on catheter tip 28. For example, voltages at electrodes E₁, E₂, E₃, and E₄ may all be measured, and since the z-coordinate for each of these electrodes is found from the depth-versus-voltage relationship determined during calibration and the x,y-coordinates of each electrode is found from fluoroscopic images captured substantially contemporaneously with the voltage measurements, well-known trigonometric relationships may be used to determine orientation of catheter tip 28.

As described above, the C-arm of fluoroscopic system 12 may be rotated into positions other than the AP (anterior/posterior) or vertical position, such orientation being as illustrated in FIG. 4 with the patient lying on a fluoroscopic table which is parallel to body-surface patch 30 and the z-coordinate perpendicular to the fluoroscopic table and aligned with electric field 34. If the C-arm is in an AP position, then the x,y plane is perpendicular to the z-axis. However, when for various reasons the C-arm is not oriented in the AP position, the plane of detector D is not perpendicular to the z-axis, and measurements of x- and y-coordinates in the plane of detector D need to be transformed in order to obtain a useful set of x,y,z-coordinates for catheter tip 28.

The computations required for such coordinate transformations are well-known to those skilled in mathematics and need not be described detail herein. For each determination of a 3D location of an electrode on catheter tip 28, the known quantities are: (1) values for x and yin the plane of detector D, (2) angle θ_{C} of the C-arm of fluoroscopic system 12, (3) position and orientation of the fluoroscopic table as provided by table data D_{T}, and (4) a value for z in the coordinate system aligned with the AP patient position. Many currently-available fluoroscopic systems such as fluoroscopic system 12 provide signals with table data D_{T} readily available to computer 16 for such computations, and when fluoroscopic table position and/or orientation D_{T} are adjusted and when C-arm angle θ_{C} is changed, appropriate coordinate transformations are updated. After such coordinate transformation, the 3D location for the electrode on catheter tip 28 is known. Measurements of more than one electrode on catheter tip 28 also then yield the 3D orientation of catheter tip 28.

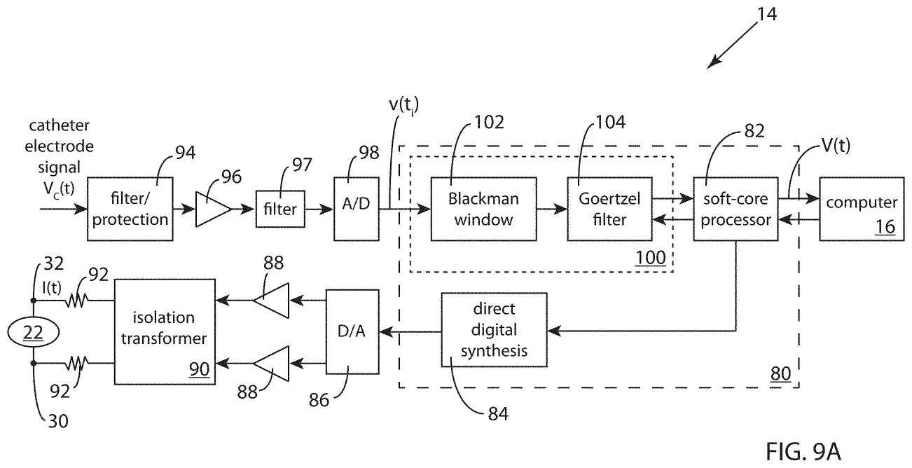

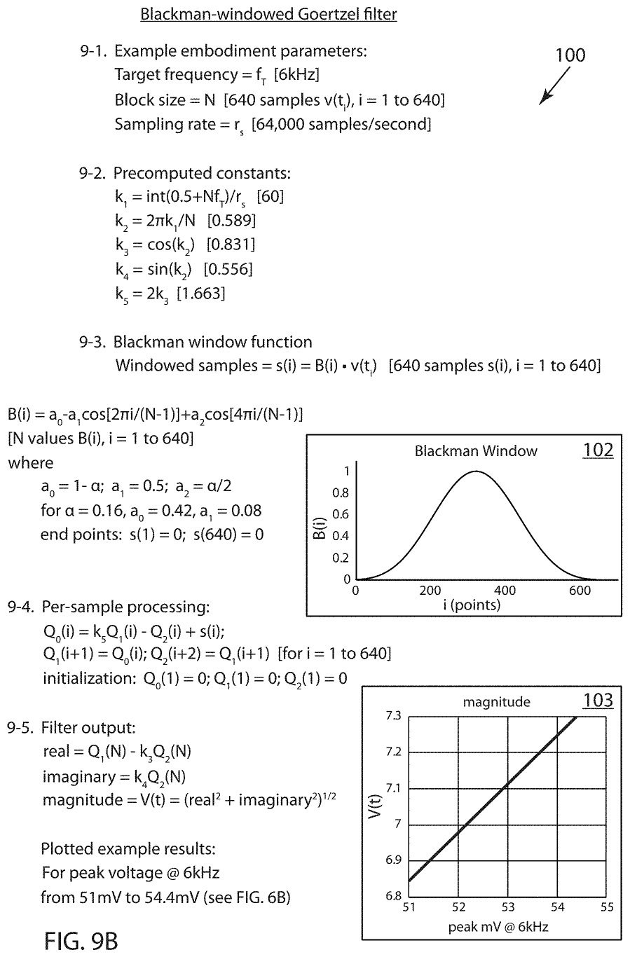

FIG. 9A is a functional block diagram of an embodiment 14 of single-axis impedance system (also referred to by reference number 14 as above) for determination of the depth coordinate of catheter tip 28 in cardiac chamber 26. As described above, an alternating current I(t) is passed through torso 22 via body-surface patches 30 and 32. In the example of FIGS. 4-6B, I(t) is a sinusoidal current having a frequency of 6 kHz and a peak amplitude of 340 μV.

In the embodiment of FIG. 9A, single-axis impedance system 14 includes an FPGA 80 (field-programmable gate array) to rapidly perform a number of computations within single-axis impedance system 14. In FIG. 9A, these computational functions are indicated as being (a) direct digital synthesis 84 of a sinusoid signal which when filtered, results in driving current I(t), (b) a Blackman window function 102 applied to a filtered and digitized catheter electrode signal v(tᵢ), (c) a Goertzel filter 104 applied to the output of Blackman window 102, and (d) a soft-core processor 82. Each of these functions will be described below. The use of FPGA 80 is not intended to be limiting; other circuit elements and programmable devices may also be used to carry out the functions realized in FPGA 80.

Driving current I(t) is generated by direct digital synthesis process 84 which produces a digitally-synthesized sinusoid of highly accurate frequency and phase. Such sinusoidal signal is then converted to an analog signal by an analog-to-digital converter 86 and buffered and filtered in buffer amplifiers 88 to smooth out the stair-step portion of the synthesized sinusoid. Finally, the filtered output from buffer amplifiers 88 passes through an isolation transformer 90 and two resistive loads 92 before being applied to torso 22 through body-surface patches 30 and 32. The result of driving current I(t) being applied across torso 22 is that due to the distribution of electrical impedance within torso 22 including cardiac chamber 26, a catheter voltage signal V_{C}(t) is created on an electrode (e.g., E₁, E₂, E₃, or E₄) on catheter tip 28 as described above with respect to FIGS. 4-6B.

Catheter voltage signal V_{C}(t) is filtered in a filter 94 which provides low- and high-pass filtering and protection to limit energy from cardiac ablation and to permit recovery from pacing and defibrillation pulses. (As shown in FIG. 5, cardiac catheter tip 28 may be the tip of a cardiac ablation catheter, and when ablation is occurring using electrode E₁, the circuitry of single-axis impedance system 14 is thereby isolated from such ablation process.)

Output from filter 94 is buffered by buffer amplifier 96, passes through a low-pass filter (set at 10 kHz, such setting not intended to be limiting) to reduce signal noise, and is then converted to a digital stream of voltage values in an analog-to-digital converter 98 as input to a Blackman-windowed Goertzel filter 100 which includes Blackman window function 102 and Goertzel filter 104. Filter embodiment 100 evaluates the digital voltage from A/D converter 98 using synchronous detection. The advantage of synchronous detection is its ability to extract low-level signals from signals which may contain a significant amount of noise. The output from A/D converter 98 is a stream of interim digital voltage values v(tᵢ) which in the example being illustrated herein, is a stream of voltage values sampled 64,000 times per second. (This sampling rate is not intended to be limiting; other appropriate sampling rates are possible.)

Filter 100 is configured to measure the signal at a specific target frequency while to a great degree ignoring portions of the signal at other frequencies, thereby measuring that portion of signal v(tᵢ) which is of most importance. Blackman window function 102 is applied as shown in section 9-3 to each of the samples v(tᵢ) in a block. Blackman-windowed Goertzel filter 100 is one example of applying synchronous detection and is not intended to be limiting; other configurations are within the scope of the present invention. For example, other window functions other than Blackman filter 102 may be combined with Goertzel filter 104, and other substantially different approaches to synchronous detection may also be employed.

FIG. 9B presents a detailed description of embodiment 100 of Goertzel filter 104 for which the input voltage has been windowed using Blackman window 102. The description of embodiment 100 is divided into five sections 9-1 through 9-5 in FIG. 9B and is described both in generality as well as referring to the exemplary values (in brackets) based on the example of FIGS. 4-6B. Section 9-1 presents parameters for the operation of embodiment 100, and section 9-2 presents a set of precomputed Goertzel-filter constants k₁ though k₅. In each application of embodiment 100, which is occurring every N/rₛ seconds, a group of N voltage values are processed as a block. In the example, a block of 640 values is processed every 0.01 seconds. (Such block size and the other parameter values of this example are not intended to be limiting; many other sets of parameters are within the scope of the present invention.)

Section 9-3 describes the application of Blackman window 102 to stream of interim digital voltage values v(tᵢ) generated by A/D converter 98. Blackman window 102 is applied to the N interim digital voltage signal values in the block of data. The use of window functions is well-known to those skilled in the art of digital filtering, and Blackman window 102 is among the set of window functions often used in the design of digital filters. The Blackman window parameter values shown in section 9-3 are close approximations to those for an exact Blackman filter. Values given here are not intended to be limiting; other sets of parameters are within the scope of the present invention.

Section 9-4 of FIG. 9B presents the per-sample computations required within Goertzel filter 104. One of the properties of Blackman window function 102 is that B(1)=B(N)=0, ensuring that sample values s(1)=0 and s(N)=0. Also, for Goertzel filter 104, initial internal filter values Q₀(1), Q₁(1), and Q₂(1) are all equal to 0. Computations for i=1 through N (640) proceed sequentially, and the filter output is computed as shown in section 9-5. Filter output is a complex quantity with real and imaginary parts as shown, and the final desired value (herein called “magnitude”) is the square root of the sum of the squares of the real and imaginary parts as shown.

Section 9-5 also includes a plot 103 which shows the results of the calculations as presented in FIG. 9B for the example as shown in FIGS. 4-6B. In this example, the magnitude is referred to as V(t), the output of single-axis impedance system 14 for catheter electrode input values of voltage from 51 mV to 54.4 mV peak values at 6 kHz. Plot 103 shows that final output V(t) is linearly related to the input voltages. Final output V(t) is a stream of digital values, one every 0.01 seconds in the example, which is provided to computer 16 for final determination of the location along the axis of single-axis impedance system 14.

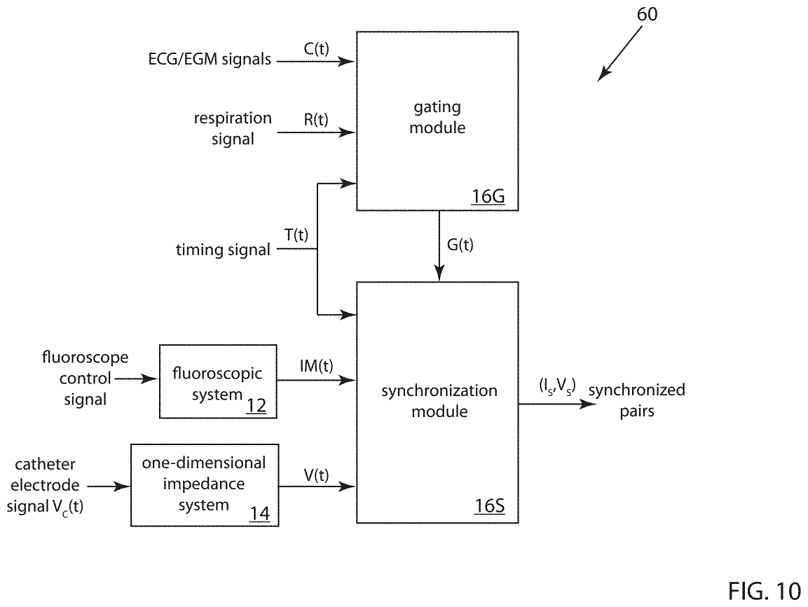

FIG. 10 is a block diagram schematic illustrating an embodiment 60 of a method for substantially contemporaneously measuring voltages from single-axis impedance system 14 and capturing images from fluoroscopic system 12. In embodiment 60, a synchronization module 16S within computer 16 (see FIG. 1) associates time reference T(t) with (a) a stream of captured fluoroscopic images I(t) from fluoroscopic system 12, (b) a stream of voltage measurements V(t) from single-axis impedance system 14, and (c) ECG/EGM signals C(t) so that every measurement of voltages V(t), signals C(t), and x,y coordinates from images I(t) share the same timing reference, thereby assuring not only that image and voltage measurements are substantially contemporaneous, but also that all of the necessary signals for procedures such as cardiac activation mapping are time-marked all based on the same timing signal T(t). The same time-marking is provided as required for respiration signal R(t). Note that in embodiment 10 of FIG. 1 and embodiment 60 of FIG. 10, the notation V(t) may represent voltages measured at more than one electrode. In other words, V(t) may be a vector quantity consisting of voltages measured from multiple electrodes. In the same way, ECG/EGM signals C(t) may also be multiple-component vector of signals.

As illustrated in embodiment 60, timing signal T(t) is an input to both a gating module 16G and synchronization module 16S and is thus the common reference for every signal (and image) in embodiment 60, including ECG/EGM signals C(t) and respiratory signal R(t) which in embodiment 60 are inputs to gating module 16G. The source of timing signal T(t) may be computer 16 or an external device such as equipment (not shown) used to capture the ECG/EGM signals C(t). Such external equipment is well-known in the field of cardiology and need not be described herein. In all cases, timing signal T(t) is essentially the master time to which all signals are referenced.

As an example to illustrate the role of time-marking of the various signals involved in the method, fluoroscopic system 12 may capture 2D images IM(t) at the rate of 7.5 fps (frames per second) or every 133 ms (milliseconds); single-axis impedance system 14 may output voltages V(t) every 10 ms, and ECG/EGM signals C(t) may stream at the rate of 1,000 sps (sample per second). In addition, respiration signals R(t) may stream at yet a different rate. Time-marking all such signals based on common timing signal T(t) assures that each of the signals is understood in its proper relationship to all of the other signals. The specific frequencies in this example set of frequencies are not intended to be limiting in any way.

In embodiment 60, in addition to establishing the substantially contemporaneous voltage measurements V(t) and image captures IM(t), fluoroscopic images IM(t) are gated with respect to both cardiac and respiratory phase to reduce motion within the fluoroscopic images which are processed to obtain x,y coordinates within the plane of X-ray detector D. Gating can be achieved by selecting images from within the stream of captured images IM(t) and/or by selectively capturing images at times when it is anticipated that gating criteria are satisfied based on cardiac signals C(t) and respiratory signal R(t).

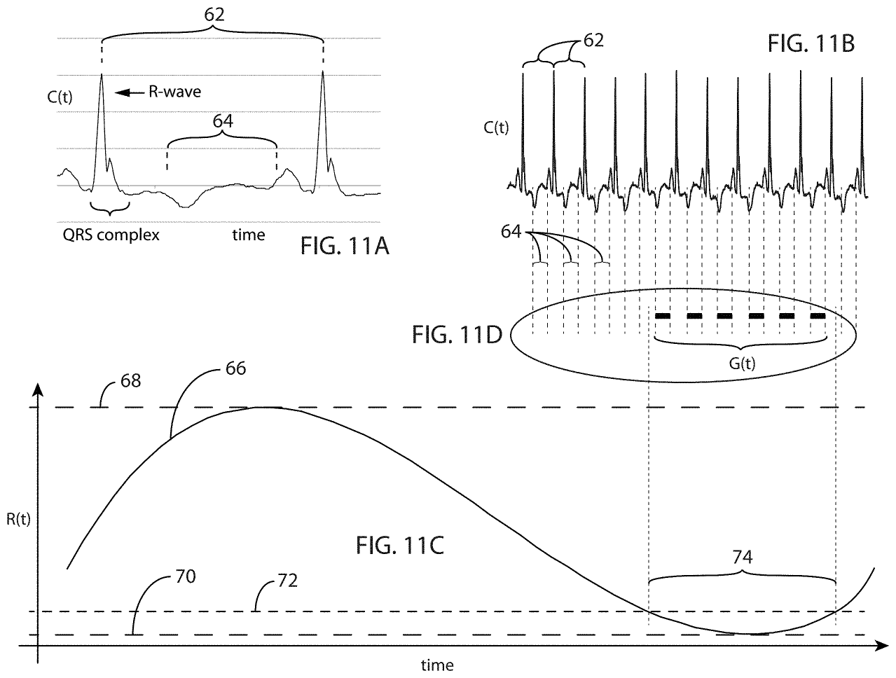

FIGS. 11A-11D are illustrations of an exemplary cardiac signal C(t) and respiratory signal R(t) being combined to generate a gating signal G(t) within gating module 16G. FIG. 11A illustrates exemplary cardiac signal C(t) illustrating two local activations (two R-waves shown). Note that as with voltage signal V(t), the notation for cardiac signal C(t) may also be representing multiple cardiac signals typically captured, and thus C(t) may be a vector signal, and the plot illustrated is one component of such vector. In the example of FIGS. 11A-11D, cardiac signal C(t) is a scalar signal as is respiratory signal R(t). Such example is not intended to be limiting.

FIG. 11B illustrates exemplary cardiac signal C(t) of FIG. 11A but with twelve local activations occurring rapidly such as when a patient is experiencing atrial fibrillation. FIG. 11C illustrates one breathing cycle of an idealized exemplary respiration signal R(t) from a sensor (not shown) for measuring respiration phase. Various sensors and techniques for capturing respiratory phase are well-known and need not be described herein.

Referring again to FIG. 11A, two QRS complexes are illustrated with an R-wave interval 62, which is the time between successive R-waves (and also the cardiac cycle length). One example of a cardiac gating criterion is illustrated, in this case, criterion 64 which is the time period within R-wave interval 62 that is between about 30% and 80% of R-wave interval 62 (during diastole) after an R-wave occurrence. FIG. 11B illustrates eleven such time periods (also labeled 64) during which gating criterion 64 is satisfied.

Referring again to FIG. 11C, respiratory signal R(t) represents respiratory movement between maximum inspiration 68 and minimum expiration 70. An exemplary respiratory gating criterion 72 is illustrated. Criterion 72 defines a period of time 74 during which respiratory phase is within a predetermined fraction of approximately 10% above minimum expiration 70 of the difference between maximum inspiration 68 and minimum expiration 70. Both cardiac criterion 64 and respiratory criterion 72 are not intended to be limiting; other values for such criteria are possible as are other forms of criteria.

FIG. 11D is a schematic representation of exemplary gating signal G(t) generated by gating module 16G by combining the results of cardiac gating and respiration gating. Gating signal G(t) as illustrated here is a series of six time periods during which both cardiac 64 and respiration 72 criteria are satisfied. The sequence of time periods which comprise gating signal G(t) represent appropriate times during which motion within the images of stream of images IM(t) is low and therefore the best opportunities for x,y coordinates within such images to be measured.

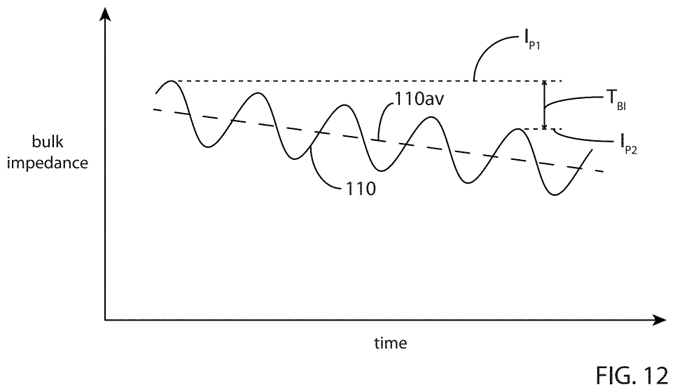

FIG. 12 is an idealized representation of the variation of bulk impedance across a portion of torso 22 of a patient as it varies due to respiration and more slowly to the addition of saline into the patient during a procedure. As shown in FIG. 4, single-axis impedance system 14 includes body-surface reference patch 36 and body-surface impedance patch 38. The bulk impedance, or transthoracic impedance, increases with inspiration, and this oscillatory variation is represented in an idealized manner by the sinusoidal character of bulk impedance plot 110. Variation of bulk impedance changes much more slowly with the addition of saline, and this variation, or drift, is represented by the average impedance 110av as shown with a dotted line.

Bulk impedance is measured by monitoring the voltage at body-surface impedance patch 38 in just the same way as measurements of catheter electrode voltages V_{C}(t). In fact, in FIG. 9A, the voltage for measuring bulk impedance can be simply an additional voltage in the vector of voltages V_{C}(t); such bulk impedance voltage is just another component in vector V_{C}(t) along with the voltages from whatever catheter electrode voltages are being measured.

As bulk impedance changes over time and such change exceeds a bulk-impedance threshold T_{BI}, the inventive method recalibrates the scale factor A. This is illustrated as the difference between peak inspiration impedance values I_{P1} and I_{P2} reaching the threshold value T_{BI}. Threshold T_{BI} may be a percentage (e.g., 10%) of the bulk impedance value I_{P1} measured after the most recent calibration. Such threshold value determination is not intended to be limiting; other indications that recalibration may be beneficial are within the scope of the present invention.

The present inventive method has a number of significant advantages when compared with current navigational systems. When compared to systems such as the CARTO® and EnSite™ NavX™ systems which use both magnetics and electrical impedance, in addition to the clear advantage of the inherent 2D accuracy of fluoroscopic images, there are a number of advantages which single-axis impedance system 14 contributes to the present inventive method. Among these are the following: (1) Single-axis impedance system 14 is easier to compensate for measurement anomalies than multi-axis impedance systems. (2) AP-oriented single-axis current path 34 (same reference number as electric field 34) is less impacted by the lungs than lateral current paths of multi-axis impedance systems. (3) AP-oriented single-axis current path 34 is the shortest path and has the lowest impedance of the three-axes across torso 22; for error represented as a percentage of the total impedance, a percentage of a smaller number results in smaller error. (4) Changes in bulk impedance over time due to drift is proportional to total impedance, resulting in lower absolute drift for the lower total impedance of the shortest axis. (5) In three-axis impedance systems, the problematic axis is the neck-to-leg axis because of the magnitude of the impedance and the propensity for movement of patches on parts of the body that can move changing the current path. Single-axis impedance system 14 avoids this axis. (6) Single-axis impedance system 14 requires fewer body-surface patches and shorter setup time, and therefore has less opportunity for setup errors and patches becoming loose.

When compared in a cardiac mapping procedure to the Navik 3D® system developed by APN Health®, LLC (described in the Sra et al. reference), it is estimated that the present inventive hybrid fluoro/impedance approach is five times more efficient than the Navik 3D® system in producing map points for a given amount of patient radiation exposure. Such combination of low radiation exposure, accuracy, and the attendant rapid speed of generating mapping points provides an important advance in medical navigational technology. For the clinical objective of generating a certain number of map points, the present inventive hybrid fluoro/single-axis impedance navigational method for determining the 3D location and orientation of a catheter tip in a patient's cardiac chamber would require one-fifth the radiation required by the Navik 3D® system.

During operation of the hybrid fluoro/single-axis impedance system, the inherent accuracy of the fluoroscopic images is used to calibrate the impedance using points at the top and bottom of a chamber rather than using the body-surface electrodes of conventional 3D impedance systems. In this way, the inventive method avoids errors introduced by non-homogeneous tissue between the body-surface patches and the cardiac chamber. Using the inventive methods of calibration provides better performance because the impedance values are pegged at or near the boundaries of the chamber and have improved linearity within the chamber because the tissue medium (blood) is relatively uniform from an electric field perspective.

Finally, and maybe most significantly, the overall speed with which a cardiac map may be generated provides a dramatic improvement. With single-axis impedance data being acquired very rapidly, it is possible to essentially generate a map point at a large fraction of the frames during cardiac diastole because the x,y coordinates of a catheter tip can be reliably determined from a single frame. Thus, extremely rapid cardiac mapping is possible using the present inventive method.

While the principles of this invention have been described in connection with specific embodiments, it should be understood clearly that these descriptions are made only by way of example and are not intended to limit the scope of the invention.

1. A method for determining the 3D location and orientation of a catheter tip in a patient's cardiac chamber, the catheter having a distal end portion including two or more electrodes adjacent thereto, the method comprising:
placing first and second body-surface patches on the patient in locations such that the cardiac chamber is therebetween, the first and second body-surface electrodes defining a depth dimension;
driving an alternating current between the patches;
measuring the voltage at the electrodes and substantially contemporaneously capturing a 2D fluoroscopic image of the cardiac chamber; and
determining the 3D location and orientation of the catheter distal end portion from the image and the measured voltages.

2. The method of claim 1 further including placing a body-surface reference patch on the patient, the voltages being measured with respect to the reference patch.

3. The method of claim 1 wherein the alternating current has a constant peak-to-peak amplitude.

4. The method of claim 1 wherein the first body-surface patch is positioned on the patient's chest, and the second body-surface patch is positioned on the patient's back.

5. The method of claim 1 wherein the step of measuring voltage includes using synchronous detection.

6. The method of claim 5 wherein the step of measuring voltage includes applying a Goertzel filter to the voltage.

7. The method of claim 6 wherein the output of the Goertzel filter is a complex number having real and imaginary parts, and the output is transformed into a real number by computing the square root of the sum of the squares of the real and imaginary parts.

8. The method of claim 7 wherein a window function is applied to the voltage prior to applying the Goertzel filter.

9. The method of claim 8 wherein the window function is a Blackman window.

10. The method of claim 1 further including correcting for changes in fluoroscopic table position and orientation and C-arm angle.

11. The method of claim 1 further including calibration steps comprising:
locating one electrode of the catheter distal end portion at two or more calibration locations within the cardiac chamber, some of the calibration locations being separated from the other calibration locations along the depth dimension;
determining spatial coordinates of the one electrode in each calibration location using only fluoroscopy;
measuring the voltages at the one electrode at each calibration location; and
computing a depth-versus-voltage relationship therefrom.

12. The method of claim 11 wherein determining the spatial coordinates of the one electrode includes capturing two 2D fluoroscopic images of the cardiac chamber from different angles and applying back-projection calculations thereto.

13. The method of claim 11 wherein computing the depth-versus-voltage relationship includes determining a linear regression relationship between the voltages and the corresponding depths of the calibration locations.

14. The method of claim 11 wherein determining the spatial coordinates of the one electrode includes the steps of:
capturing a stream of digitized 2D images of the cardiac chamber from a single angle;
detecting an image of the one electrode in a subset of the digital 2D images;
applying to the digital 2D images calculations which preserve original pixel intensity values and permit statistical calculations thereon, using a plurality of unfiltered raw-data cross-sectional intensity profiles and statistically combining the profiles to estimate image dimensions, thereby to measure the electrode image;
applying conical projection and radial elongation corrections to the image measurements; and
calculating the spatial coordinates of the electrode from the corrected 2D image measurements.

15. The method of claim 1 further including placing a body-surface impedance-monitoring patch on the patient, measuring the voltage thereon, and monitoring bulk impedance of the patient.

16. The method of claim 15 further including the step of recalibration when a change in the bulk impedance exceeds a threshold.

17. The method of claim 1 wherein measuring the voltages and capturing the 2D fluoroscopic images are gated by respiratory phase.

18. The method of claim 1 wherein measuring the voltages and capturing the 2D fluoroscopic images are gated by cardiac phase.

19. The method of claim 18 wherein measuring the voltages and capturing the 2D fluoroscopic images are gated by respiratory phase.

20. The method of claim 1 wherein one of the two or more electrodes is an ablation electrode, and the ablation electrode is electrically-isolated from voltage measurement circuitry during ablation.

21. The method of claim 1 further including capturing ECG/EGM signals from the patient and time-marking the measured voltages, the captured 2D fluoroscopic image, and the ECG/EGM signals with a common timing signal.

22. The method of claim 21 further including time-marking a respiration signal with the common timing signal.

23. A method for determining the 3D location of a catheter distal end in a patient's body, the distal end including an electrode, the method comprising:
placing first and second body-surface patches on the patient in positions such that body region of interest is therebetween;
driving an alternating current between the patches;
measuring the voltage at the electrode and substantially contemporaneously capturing a 2D fluoroscopic image of the region of interest; and
determining the 3D location of the catheter distal end from the image and the measured voltage.
